(19) United States
(12) Patent Application Publication
(10) Pub. No.: US 2013/0295179 A1
(43) Pub. Date: Nov. 7, 2013

(54) NOVEL OMEGA-3 AND OMEGA-6 FATTY ACID COMPOSITIONS AND USES THEREOF

(71) Applicants: Christian TERREAUX, Chardonne (CH); Eleonora HOMBERGER, Aesch (CH); Burkhard KRIWET, Grenzach-Wyhlen (DE); Eduardo BARRAGAN PEREZ, Valle de las Palmas (MX)

(72) Inventors: Christian TERREAUX, Chardonne (CH); Eleonora HOMBERGER, Aesch (CH); Burkhard KRIWET, Grenzach-Wyhlen (DE); Eduardo BARRAGAN PEREZ, Valle de las Palmas (MX)

(21) Appl. No.: 13/862,179

(22) Filed: Apr. 12, 2013

Related U.S. Application Data
(63) Continuation-in-part of application No. PCT/EP2011/067844, filed on Oct. 12, 2011.
(60) Provisional application No. 61/445,736, filed on Feb. 23, 2011.
(60) Provisional application No. 61/392,106, filed on Oct. 12, 2010.

(51) Int. Cl. A61K 31/202; A61K 33/26; A61K 33/30; A61K 31/4458; A61K 45/06

(52) U.S. Cl. CPC A61K 31/202; A61K 31/4458; A61K 45/06; A61K 33/30; A61K 33/26
USPC 424/479; 514/317; 514/52; 514/167; 514/249; 514/89; 424/643; 424/646; 424/474; 424/48

(57) ABSTRACT

The present application relates to compositions for use in a method of treating and/or preventing mental and behavioral disorders and/or improving mental health and cognitive functions in a subject, comprising:
a) Docosahexaenoic acid (DHA) and/or the pharmaceutically acceptable derivatives and/or precursors thereof;
b) Eicosapentaenoic acid (EPA) and/or the pharmaceutically acceptable derivatives and/or precursors thereof;
c) γ-linolenic acid (GLA) and/or the pharmaceutically acceptable derivatives and/or precursors thereof; and
   at least a psychostimulant compound selected among methylphenidate HCl, atomoxetine, amphetamine, gamma-hydroxybutyrate, dextroamphetamine, sibutramine, methylenedioxymethamphetamine, and/or an anxiolytic compound selected among fluoxatine, sertraline, paroxetine, fluoroxamine, citralopram, venlafaxine, bupropion, nefazodone, and mirtazapine.

The present application also relates to the method of treating and/or preventing mental and behavioral disorders and/or improving mental health and cognitive functions in a subject, comprising administering an effective amount of a composition comprising:
a) Docosahexaenoic acid (DHA) and/or the pharmaceutically acceptable derivatives and/or precursors thereof;
b) Eicosapentaenoic acid (EPA) and/or the pharmaceutically acceptable derivatives and/or precursors thereof;
c) γ-linolenic acid (GLA) and/or the pharmaceutically acceptable derivatives and/or precursors thereof; and
   at least a psychostimulant compound selected among methylphenidate HCl, atomoxetine, amphetamine, gamma-hydroxybutyrate, dextroamphetamine, sibutramine, methylenedioxymethamphetamine, and/or an anxiolytic compound selected among fluoxatine, sertraline, paroxetine, fluoroxamine, citralopram, venlafaxine, bupropion, nefazodone, and mirtazapine.

### FIELD OF THE INVENTION

The present invention provides novel compositions comprising combinations of omega-3 and omega-6 fatty acids, with a psychostimulant and/or anxiolytic compound, and optionally with at least one vitamin and one or more minerals. These compositions are particularly useful for treating and/or preventing mental and behavioral disorders and/or improving mental health and cognitive functions either in healthy subjects or in subjects in need of such treatment.

### BACKGROUND OF THE INVENTION

Polyunsaturated fatty acids (PUFAs) are important for all humans in general, and children and women specifically. They are concentrated in the brain and nervous system. Docosahexaenoic acid (DHA) (omega-3) is the most abundant polyunsaturated fatty acid in the brain, where it is concentrated in the neuronal synapses and appears to be integral to neurotransmission. Eicosapentaenoic acid (EPA), Arachidonic (AA) acid, and gamma-linolenic acid (GLA) (omega-6) are also considered to be important in neurotransmission and deficiencies of these fatty acids have been linked with psychiatric and developmental disorders. The association between deficiencies in omega-3 and omega-6 fatty acids and children's behavior and learning difficulties has been established in a number of studies. These neuro-developmental disorders include hyperkinetic disorders such as Attention Deficit Hyperactivity Disorder (ADHD) or Attention Deficit Disorder (ADD), hyperactivity, or cognitive dysfunction and disorders of psychological development, such as autism or Autistic syndrome disorder (ASD), apraxia, dyspraxia, or dyslexia in school-age children and young adults. Omega-3 and omega-6 cannot be synthesized by humans and must be provided by means of dietary sources. These dietary sources, however, have been declining in Western societies. In 20% of children with behavioral and learning difficulties, standard interventions, such as targeted reading practice, structured coordination activities, and behavioral management, have resulted in only minimal improvements.

A number of reports and studies have been conducted on the effect of fatty acid supplements on ADHD. Most of these earlier reported studies merely create a doubt on the effectiveness of fatty acid supplementation on ADHD. For example, Alternative Medicine Review Volume 12, Number 3 2007, Parris M. Kidd, PhD reported that Clinical evidence from controlled trials, open studies, and case reports has yielded mixed results from DHA/EPA supplementation in ADHD and its co-morbid conditions. Transler C et al. (J Atten Disord. 2010 Apr. 27) also concluded that evidence on effect of fatty acid supplementation on ADHD is too limited to reach definitive conclusions but suggested that research on the impact of long-chain PUFA (n-3 and n-6) should continue. The study of Sinn N. and Bryan J. (J Dev Behav Pediatr 2007; 28:82-91) involved 132 Australian children aged 7-12 years who scored SD above population norms on the Conners' ADHD index, a subscale of the Conners' Parent Rating Scale-Long Version (CPRS-L), but who did not have a clinical diagnosis of ADHD for the study. The children underwent randomization to receive 3 months of omega-3/-6 supplementation or matching placebo. A micronutrient supplement containing vitamins (A, B₂, B₃, B₅, B₆, B₁₂, C, D₃, and E) and minerals (zinc, magnesium, copper, and iron), or placebo, was added to assess if supplementation yielded any additional benefit. Significant reductions in ADHD symptoms were found on the CPRS-L, but not on the corresponding CTRS-L, and there was no significant difference with or without micronutrients. In yet another study published in J. of Att. Dis. 2009; 12 (5) 394-401, only a subgroup of children and adolescents with ADHD treated with omega 3/6 fatty acids for 6 months responded with meaningful reduction of ADHD symptoms. Hence there is still a need to develop effective fatty acid combinations that would prove to be superior to the prior known supplements.

Omega-3 and omega-6 polyunsaturated fatty acids (PUFAs) play a central role in the normal development. They are integral parts of the neuronal cell membranes in the brain and it has been hypothesized that they may have a role in facilitating the transmission of signals between neurons. It has been observed that imbalances and deficiencies may have an impact not only on the development and but also on the brain functioning in such a way, that manipulation of these PUFAs may have repercussions in this regard. The adequate presence of certain polyunsaturated fatty acids as eicosapentaenoic acid (EPA), docosahexaenoic acid (DHA) and gamma-linolenic (GLA) and the omega-3/omega-6 ratio are fundamental in the treatment of some neurodevelopment disorders. Consequently, intervention studies conducted in children affected by ADHD and learning disabilities were designed to investigate whether combinations of different PUFAs play a significant role in relieving the symptoms.

Attention-Deficit Hyperactivity Disorder (ADHD) is the most frequently observed psychiatric disorder of childhood, which causes failure at school and serious behavioural problems in children. ADHD has a worldwide prevalence of 5.28%. In Latin America it affects between 3% and 7% of the population and is considered as a public health problem. ADHD is most predominant in children of school age and is usually more common among boys than girls. It is characterised by the presence of different degrees of lack of attention, hyperactivity and impulsivity which impact on the child's overall conduct, notably with regard to development at school.

Functional impairment is the most important factor in ADHD. Treatment is multimodal and the psychopharmacological approach constitutes the cornerstone of the treatment. Stimulants or psychostimulants represent the group of most-used medications, among them methylphenidate (MTP). There are various pharmacological options for ADHD, on its own or with co-morbidity, such as non-stimulant medications (atomoxetine, tricyclic antidepressants, alpha-adrenergic agonists and modafinil). Nutritional therapies in which the ingestion of certain groups of food is restricted (fatty acids, sugars, food containing salicylates, etc.), or the ingestion of high doses of vitamins, dietary minerals (cadmium, copper, iron, zinc) or omega-3/6 is recommended. It has been put forward that, in exceptional circumstances, deficiency in long-chain polyunsaturated fatty acids may be involved in the development of the attention-deficit hyperactivity disorder and that the extra contribution may be of some benefit with regard to some inherent features of ADHD and other behavioural disorders. After the establishment of the relationship between hyperactivity and polyunsaturated fatty acid deficiency multiple trials with supplementation of a combination of the omega-3 fatty acids, eicosapentaenoic acid (EPA) and docosahexaenoic acid (DHA) and the omega-6 fatty acid gamma-linolenic acid (GLA) have been performed. These trials reported significant symptom reductions in several neurodevelopmental disorders as ADHD total scores, ADHD inattentive scores, reading-writing disorders, dyspraxia, motor coordination difficulties. This combination has been by far the one which has shown the best research results for ADHD symptoms.

The search for alternatives to pharmacological treatments in resolving attention problems, with fewer side-effects is a priority in Latin-American countries. In Mexico there are no clinical studies with supplements that show improvement in the symptoms of the attention-deficit hyperactivity disorder, and to see whether the Latin-American population responds as appropriately as in other parts of the world. The aim of the study was to assess the efficacy and safety of the use of omega-3/6 on its own and in combination with methylphenidate for the treatment of symptoms of ADHD in a Mexican child population.

### SUMMARY OF THE INVENTION

According to a first embodiment, the present invention relates to a composition for use in a method of treating and/or preventing mental and behavioral disorders and/or improving mental health, learning abilities and/or cognitive functions in a subject comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof,
   in combination with analeptic agents or psychostimulants, and/or with anxiolytic compounds.

The present invention also relates to methods of treating and/or preventing mental and behavioral disorders, and/or improving mental health, learning abilities and/or cognitive functions a subject comprising administering a composition comprising an effective amount of
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof, and
   in combination with analeptic agents or psychostimulants, and/or with anxiolytic compounds.

The present invention further relates to compositions comprising a combination of omega-3 and omega-6 fatty acids with the standard care of Attention Deficit Hyperactivity Disorder (ADHD) for use in a method of treating and/or preventing learning abilities and/or cognitive functions of ADHD children. Compositions of the present invention are in particular beneficial for treating ADHD patients and for reducing secondary adverse events generally encountered when using standard dose of the ADHD standard care.

The compositions according to the present invention are preferably administered orally to a healthy human subject or a human subject in need of such treatment, as a food supplement or as a food for special medical purposes.

According to one aspect, the present invention provides a food supplement comprising the foregoing compositions, and thus can be administered as a food supplement and/or a food for special medical purposes. In another aspect, the present invention provides a pharmaceutical composition comprising the foregoing compositions, specifically said composition is in the form of a pharmaceutical composition, and comprises a pharmaceutically acceptable vehicle.

### BRIEF DESCRIPTION OF THE FIGURES

FIG. 1 relates to the study design, showing a schematic depiction of the different study periods (MTP: Methylphenidate; EPA: Eicosapentaenoic acid; DHA: Docosahexaenoic acid; GLA: gamma-linolenic acid).

FIG. 2 show shows the randomization done across patient dispositions.

FIG. 3 shows MTP doses of group A and group C during treatment period. Group A: MTP; Group C Omega3/6+MTP; **: p<0.001.

FIG. 4 shows mean changes in ADHD-RS-SV at baseline and after 1 month and 3, 6, and 12 months for all 3 treatment groups. Group A: MTP; Group B: Omega 3/6; Group C: Omega3/6+MTP; **: p<0.001.

FIG. 5 shows mean changes in Investigator rated CGI at baseline and after 1 month and 3, 6, and 12 months for all 3 treatment groups. Group A: MTP; Group B: Omega 3/6; Group C: Omega3/6+MTP; **: p<0.01 for the measurement between the three groups after 12 months. P<0.001 at every measurement time compared to respective baseline for all groups.

### DETAILED DESCRIPTION OF THE INVENTION

The present invention is directed to methods of treatment and compositions that combine the nutritional, health and/or medical benefits provided to subjects by omega-3 fatty acids and omega-6 fatty acids with defined analeptic agents or psychostimulants, and/or with anxiolytic compounds, and optionally with selected minerals and vitamins. The present invention also relates to methods for producing such compositions, and to methods for using such compositions to provide one or more nutritional, health and/or medical benefits to a subject, or to enhance one or more of such benefits in a subject.

The present inventors have surprisingly found this novel composition based on a novel combination of elements, to be highly effective for use in treating and/or preventing mental and behavioral disorders and/or improving mental health and/or cognitive functions in a subject.

Specifically it has been found that the novel compositions of the present invention are effective for use in method of treating and/or preventing neuropsychiatric or psychiatric disorders such as hyperactivity, anxiety, depression, schizophrenia, or bipolar disorders, hyperkinetic disorders such as Attention Deficit Hyperactivity Disorder (ADHD) or Attention Deficit Disorder (ADD), hyperactivity, or cognitive dysfunction, disorders of psychological development, such as autism or Autistic syndrome disorder (ASD), apraxia, dyspraxia, or dyslexia in children and adults. ADHD is a preferred disorder to be treated.

In the last few years, psychiatrists have realized that ADHD is not only a disorder of childhood, but often continues in the adult. It is obvious that hyperactivity and short attention span cause grave disruption in an adult's life. Adults with attention deficit/hyperactivity disorder may have difficulty following directions, remembering information, concentrating, organizing tasks or completing work within time limits. If these difficulties are not managed appropriately, they can cause associated behavioral, emotional, social, vocational and academic problems. The novel composition of the present invention is also beneficial for adult subjects suffering from hyperactivity.

The compositions of the present invention are found particularly effective in improving learning abilities and/or cognitive functions in normal healthy school-age children and young adults with average behavior and intelligence. In particular the compositions of the invention are also effective in improving the signs of cognitive performance or decreasing the symptoms of cognitive dysfunction.

The present invention thus provides compositions including unique combinations of omega-3 and omega-6 fatty acids in specific ratios in combination with defined psychostimulants and/or anxiolytics compound, and optionally with vitamins and/or minerals. In particular, the present compositions comprise effective amount of omega-3 and omega-6 fatty acids, preferably eicosapentaenoic acid (EPA), docosahexaenoic acid (DHA), γ-linolenic acid (GLA).

“Fatty acids” refer to a family of carboxylic acids having a hydrocarbon chain of from about 12 to about 24 carbons in length. Unsaturated fatty acids have at least one carbon-carbon double bond in the hydrocarbon chain. Unsaturated fatty acids include monounsaturated fatty acids and polyunsaturated fatty acids (PUFAs). Unsaturated fatty acids are designated by the position of the first double bond from the methyl end of the hydrocarbon chain. Omega-3 fatty acids have a first double bond at the third carbon from the methyl end of the chain; and include, e.g., α-linolenic acid (octadeca-9,12,15-trienoic acid), stearidonic acid (octadeca-6,9,12,15-tetraenoic acid), eicosapentaenoic acid (eicosa-5,8,11,14,17-pentaenoic acid; “EPA”), docosapentaenoic acid (docosa-7,10,13,16,19-pentaenoic acid), eicosatetraenoic acid (eicosa-8,11,14,17-tetraenoic acid), and docosahexaenoic acid (docosa-4,7,10,13,16,19-hexaenoic acid; “DHA”). Omega-6 fatty acids have a first double bond at the sixth carbon from the methyl end of the chain; and include, e.g., linoleic acid (9,12-octadecadienoic acid), γ-linolenic acid (6,9,12-octadecatrienoic acid; GLA), eicosadienoic acid (11,14-eicosadienoic acid), dihomo-γ-linolenic acid (8,11,14-eicosatrienoic acid), arachidonic acid (5,8,11,14-eicosatetraenoic acid), docosadienoic acid (13,16-docosadienoic acid), adrenic acid (7,10,13,16-docosatetraenoic acid), docosapentaenoic acid (4,7,10,13,16-docosapentaenoic acid), and calendic acid (8E,10E,12Z-octadecatrienoic acid), and the like. The present invention also contemplates the use of precursors and derivatives of such fatty acids.

Natural sources such as fish oil are the major sources of omega-3 fatty acids, such as EPA and DHA. There are however other marine source, such as algae and krill. Omega-6 fatty acids may also be obtained from fish oil, but a major source of GLA may be primrose oil, or other vegetable sources, such as borage oil, black current seed oil.

Preferably, the fatty acids according to the present invention may be obtained by extraction, concentration and purification processes starting from fish oils for DHA and EPA, and from primrose oil typically for GLA, as well as by means of semi-synthetic transformation processes, when required.

The fatty acids are derived from fish oil by a refining process:

1. Washing: The crude oil is washed with NaOH to remove free fatty acids and with citric acid to remove water soluble proteins;
2. Winterization: saturated fats are removed by winterization, which comprises a cooling step down to below zero;
3. Bleaching: contaminants are removed by addition of bleaching clay and subsequent filtration;
4. Deodorisation: remaining volatiles which give the fishy taste, are removed, e.g. by steam-washing (the oil is put into near-vacuum and blasted with steam of about 20 atm); For highly concentrated oils, some additional steps are carried out:
5. Esterification: the triacylglycerols are saponified with alcohol and ethyl esters are formed;
6. Molecular distillation: unwanted fatty acids (short chain FA) are removed by distillation;
7. Re-esterification: triacylglycerols are reconstituted by mixing the ethyl esters with glycerol at elevated temperatures.
The specified concentrations of the individual products are achieved by mixing natural oil with the re-esterified concentrates in an appropriate ratio.

The relative amounts of the omega-3 fatty acids are preferably in the range of 1 part DHA to 1 to 10 parts (weight per weight (w/w)) EPA, in particular 1 part DHA to 2 to 5 parts (w/w) EPA, such as 1 part DHA to 3 to 3.5 parts (w/w) EPA. The relative amount of the omega-6 fatty acid GLA to the combined omega-3 fatty acids DHA and EPA are preferably in the range of 1 part GLA to 5 to 20 parts (w/w) DHA/EPA, in particular 1 part GLA to 10 to 15 parts (w/w) DHA/EPA, such as 1 part GLA to 11 to 13 parts (w/w) DHA/EPA.

Most preferred synergistic compositions according to this second embodiment comprise an increased concentration of EPA, DHA, and GLA. More particularly, a preferred ratio of EPA:DHA:GLA is equal to around (about) 9:3:1. Thus the ratio may be, or be around (about) 9:2.5-3.5:0.8-1.2. In one embodiment the ratio is, or is around (about) 9:2.8-3.2:0.9-1.1.

The compositions of the invention may thus comprise between 30 to 50 mg of EPA, preferably 40 to 45 mg, and most preferably around 42 mg of EPA; between 200 to 400 mg of DHA, preferably between 250 and 350 mg of DHA, preferably around 300 mg of DHA; and between 10 to 20 mg of GLA, preferably between 12 to 17 mg of GLA, and most preferably around 15 mg of GLA. Most preferred compositions thus comprise around 42 mg of EPA, around 300 mg of DHA, and around 15 mg of GLA. The composition may comprise from 5 to 50 mg of stimulant and/or anxiolytic, such as 10 to 30 mg.

Analeptic agents or psychostimulants which are used in the combinations according to the present invention include for standard care of ADHD, such as methylphenidate HCl, atomoxetine, and more generally stimulants such as amphetamine, gamma-hydroxybutyrate, dextroamphetamine, sibutramine, and methylenedioxymethamphetamine. Anxiolytic compounds may be without any limitations fluoxatine, sertraline, paroxetine, fluoroxamine, citralopram, venlafaxine, bupropion, nefazodone, and mirtazapine.

Typically the psychotimulant and/or anxiolytic are administered at a dosage of 0.1 to 2 mg/kg/day, such as at 0.5 to 1 mg/kg/day. In one embodiment the composition of the invention is administered over at least 30 days, such as over at least 60 days, or over at least 120 days.

The compositions according to the present invention may optionally comprise at least one vitamin and one or more minerals. Vitamins which may be incorporated in the present compositions may be water-soluble vitamins or oil-soluble vitamins. They include without any limitations vitamin of the B group, such as vitamin B1 (thiamin hydrochloride, thiamin mononitrate, thiamine monophosphate chloride, and thiamine pyrophosphate chloride), vitamin B2 (riboflavin and riboflavin 5′-phosphate sodium), niacin (nicotinic acid, nicotinamide, and inositol hexanicotinate), vitamin B5 (panthothenic acid), vitamin B6 (pyridoxine hydrochloride, pyridoxine 5′-phosphate, and pyridoxal 5′-phosphate), folic acid or of reduced folate (including but not limited to L-methylfolate; L-5-methyltetrahydrofolate; pteroylmonoglutamic acid; calcium-L-methylfolate; L-5-methyl-tetrahydrofolic acid; (6S) tetrahydrofolic acid; (6S)-5-methyl-tetrahydrofolic acid; 5-methyl-(6S)-tetrahydrofolic acid; 5-formyl-(6S)-tetrahydrofolic acid; 10-formyl-(6R)-tetrahydrofolic acid; 5,10-methylene-(6R) tetrahydrofolic acid; 5,10-methenyl-(6R)-tetrahydrofolic acid; 5-formimino-(6S)-tetrahydrofolic acid, and polyglutamyl derivatives of tetrahydrofolate acid); vitamin B12 (cyanocobalamin, cobalamin, hydroxocobalamin, 5′-deoxyadenosylcobalamin, and methylcobalamin), vitamin H (biotin), vitamin C (ascorbic acid, sodium-L-ascorbate, calcium-L-ascorbate, potassium-L-ascorbate, L-ascorbyl 6-palmitate, magnesium L-ascorbate, and zinc L-ascorbate), vitamin E (D-alpha-tocopherol, DL-alpha-tocopherol, D-alpha-tocopheryl acetate, DL-alpha-tocopheryl acetate, D-alpha-tocopheryl acid succinate, mixed tocopherols, and tocotrienol tocopherol), vitamin A, vitamin D (cholecalciferol and ergocalciferol), panthotenic acid (D-pantothenate, calcium, D-pantothenate, sodium, dexpanthenol, and pantethine), and/or biotin (D-biotin). Preferred compositions of the present invention comprise vitamin B5.

These vitamins are incorporated in the compositions of the present invention in amounts so as to provide to the subject the recommended daily allowance (RDA) of vitamins as defined by the WHO and multiples thereof or to provide the reference nutrient Intake (RNI) for vitamins as defined by the Food and Agriculture Organization (FAO) and multiples thereof.

By way of examples, minerals which may be incorporated in the compositions of the present invention may derived from various sources, such as iron source (ferrous sulfate, ferrous fumarate, Fe(III)-hydroxy-polymaltose complex, i.e., Maltofer®, ferrous carbonate, ferrous citrate, ferric ammonium citrate, ferrous gluconate, ferrous fumarate, ferric sodium diphosphate, ferrous lactate, ferric diphosphate, ferric saccharate, ferrous bisglycinate, ferrous L-pidolate, ferrous phosphate, and iron (II) taurate), phosphorus source, magnesium source (magnesium oxide), zinc source (zinc oxide, zinc sulfate, zinc lactate, zinc acetate, zinc L-ascorbate, zinc L-aspartate, zinc bisglycinate, zinc chloride, zinc citrate, zinc gluconate, zinc L-lysinate, zinc malate, zinc mono-L-methionine sulphate, zinc oxide, zinc carbonate, zinc L-pidolate, and zinc picolinate), selenium (sodium selenate or any other pharmaceutically acceptable form or derivative of selenium, such as L-selenomethionine, selenium enriched yeast, selenious acid, sodium hydrogen selenite, and sodium selenite), copper (cupric oxide or any other pharmaceutically acceptable form or derivative of copper, such as cupric carbonate, cupric citrate, cupric gluconate, cupric sulphate, copper L-aspartate, copper bisglycinate, copper lysine complex, and copper (II) oxide), manganese (manganese sulfate or any other pharmaceutically acceptable form or derivative of manganese, such as manganese ascorbate, manganese L-aspartate, manganese bisglycinate, manganese carbonate, manganese chloride, manganese citrate, manganese gluconate, manganese glycerophosphate, manganese pidolate, and manganese sulphate), chromium (chromium chloride or any other pharmaceutically acceptable form or derivative of chromium, such as chromium (III) lactate trihydrate, chromium nitrate, chromium picolinate, and chromium (III) sulphate), molybdenum (sodium molybdate and/or any other pharmaceutically acceptable form or derivative of molybdenum, such as ammonium molybdate: molybdenum VI, potassium molybdate: molybdenum VI, and sodium molybdate: molybdenum VI), fluoride (calcium fluoride, potassium fluoride, sodium fluoride, and sodium monofluorophosphate), iodine, calcium, boron, various mixtures and/or combinations thereof.

Preferred compositions of the present invention comprise a source of iron and/or zinc. The zinc source is preferably selected from a group comprising zinc oxide, zinc sulfate, zinc lactate, zinc acetate, zinc L-ascorbate, zinc L-aspartate, zinc bisglycinate, zinc chloride, zinc citrate, zinc gluconate, zinc L-lysinate, zinc malate, zinc mono-L-methionine sulphate, zinc oxide, zinc carbonate, zinc L-pidolate, and zinc picolinate. Also, the iron source is preferably selected from a group comprising ferrous sulfate, ferrous fumarate, Fe(III)-hydroxy-polymaltose complex, i.e., Maltofer®, ferrous carbonate, ferrous citrate, ferric ammonium citrate, ferrous gluconate, ferrous fumarate, ferric sodium diphosphate, ferrous lactate, ferric diphosphate, ferric saccharate, ferrous bisglycinate, ferrous L-pidolate, ferrous phosphate, and iron (II) taurate.

The vitamins and/or minerals are, optionally, present in a solid-state form, such as an amorphous powder or a milled material, for example, a finely milled crystalline material, but can also be present in an aqueous or other solution that is a continuous or disperse phase of an emulsion including the one or more edible oils. Water-soluble vitamins and/or minerals present in solid form in compositions of the invention preferably become at least temporarily suspended in, and coated with, the one or more edible oils.

The present invention thus provides compositions for use in a method of treating and/or preventing mental and behavioral disorders and/or improving mental health and/or cognitive functions in a subject, comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof, and
   at least an effective amount of standard care of ADHD, such as methylphenidate HCl, atomoxetine, and/or an effective amount of psychostimulant, such as amphetamine, gamma-hydroxybutyrate, dextroamphetamine, sibutramine, and methylenedioxymethamphetamine, and/or an effective amount of an anxiolytic compound, such as fluoxatine, sertraline, paroxetine, fluoroxamine, citralopram, venlafaxine, bupropion, nefazodone, and mirtazapine.

The terms “subject” and “patient” used interchangeably herein, refer to a mammal, e.g., a human. Such human subject herein includes children (school-aged children having between 5 and 12 years old) and young adults (having about between 18 to 25 years old), either normal or mainstream and/or with underlying disease conditions as defined above. The subject may have or be at risk of any of the conditions mentioned herein. The subject may have been diagnosed with any of the conditions mentioned herein. In one embodiment the subject has a specified condition as mentioned herein, and does not suffer from any other conditions.

It is believed that compositions of the invention provide mammals including children, and young adults either normal, mainstream and/or with underlying disease conditions with a superior heath benefit in terms of mental health and cognitive functions. The novel compositions may combine the active ingredients in different ways and concentrations to make them suitable for administration to children and young adults.

“Cognitive functions” or “Signs of cognitive performance” or “symptoms of cognitive dysfunction” as used herein may be selected from a group comprising fine motor skills (like cutting, writing etc.), gross motor skills (running, jumping etc.), motor coordination abilities (e.g., hand-eye coordination, like holding a pencil or buttoning a shirt etc. . . . ), learning abilities (like sequencing, memory, organization etc.,), language-based learning abilities, reading abilities (understanding the relationship between sounds, letters and words, letter and word recognition, understanding words and ideas, reading speed and fluency, general vocabulary skills etc.) writing abilities (neatness and consistency of writing, accurately copying letters and words, spelling consistency, writing organization and coherence etc. . . . ), auditory processing skills, visual perception skills, social and emotional skills, ability to complete task, ability to stay on task, ability to follow instructions, ability to complete assignments, long term memory, short term memory, ability to make a decision, ability to follow through on decision, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information etc. . . .

As used herein, the terms “treatment and/or prevention” “treating and/or preventing” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) reducing the incidence and/or risk of relapse of the disease during a symptom-free period; (b) relieving or reducing a symptom of the disease; (c) preventing the disease from occurring in a subject which may be predisposed to the disease but has not yet been diagnosed as having it; (d) inhibiting the disease, i.e., arresting its development (e.g., reducing the rate of disease progression); (e) reducing the frequency of episodes of the disease; and (f) relieving the disease, i.e., causing regression of the disease.

In particular, the novel compositions of the present invention are believed to be particularly effective for use in a method of treating and/or preventing hyperkinetic disorders and/or disorders of psychological development and/or neurodevelopment-related diseases.

Especially for treatment of abnormalities related to the central nervous system, although therapeutic interventions have been used to address individual aspects of impaired neurological function, it is desirable to provide a comprehensive holistic approach for the treatment. When complex, interrelated disease pathways are involved in an abnormal physiologic condition, a single agent does not provide significant beneficial action. Multiple sites of intervention are mandatory. It is an important feature of the invention that the components act together to provide a synergistic effect by effecting different pathways of action, for example, by normalizing the several neurotransmitters and receptor sites responsible for ADHD.

According to one embodiment, the present invention relates to the According to one embodiment, the present invention relates to a composition for use in improving learning abilities and/or cognitive functions, and/or for treating and/or preventing learning disabilities and cognitive dysfunctions in normal children and young adults or to children and young adults in need of such treatment, comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof, and
   at least a stimulant or psychostimulant compound selected among methylphenidate HCl, atomoxetine, amphetamine, gamma-hydroxybutyrate, dextroamphetamine, sibutramine, methylenedioxymethamphetamine, and/or an anxiolytic compound selected among fluoxatine, sertraline, paroxetine, fluoroxamine, citralopram, venlafaxine, bupropion, nefazodone, and mirtazapine.

These compositions are administered to normal children and are particularly beneficial for treating and/or preventing behavioral and emotional disorders. Such disorders include hyperkinetic disorders, conduct disorders, disorders of social functioning, tic disorders, and feeding disorders. Hyperkinetic disorders are disturbance of activity and attention, such as Attention Deficit Hyperactivity Disorder (ADHD) or Attention Deficit Disorder (ADD), hyperactivity, or cognitive dysfunction. Behavioral and emotional disorders are disorders of psychological development and also include disorders of speech and language, specific developmental disorders of scholastic skills, specific developmental disorder of motor function, and pervasive developmental disorders. By way of examples, we may cite autism or Autistic Syndrome Disorder (ASD), apraxia, dyspraxia, or dyslexia.

Hyperkinetic disorders correspond to disturbance of activity and attention, such as Attention Deficit Hyperactivity Disorder (ADHD) or Attention Deficit Disorder (ADD), hyperactivity, or cognitive dysfunction. These are psychiatric conditions and more specifically chronic neurobehavioral syndrome. ADHD manifests as inattention, impulsivity, and hyperactivity. ADD is a psychiatric disorder similar to ADHD but without the hyperactivity symptom. People with ADHD or ADD tend to overlook details and to miss information. Because of attention deficit, people with ADHD or ADD become averse to tasks that require concentration, decisiveness, and organizational skills and may avoid situations where they are expected to perform or to take responsibility for their effort. Attention-Deficit/Hyperactivity Disorder is one of the most important causes of scholar failure and conduct disorder in infants. This is the most common neuropsychiatric disorder in pediatric population worldwide.

ADHD and cognitive dysfunction patients and members of their family may be predisposed to developing other various other disease conditions. The present invention is expected to be useful for treating and/or reducing the risk of developing these and other diseases which may be associated with ADHD and cognitive dysfunction.

There are no biological markers neither concluding evidence for its diagnosis; this is based on clinical judgment of the DSM-IV or ICD-10, helped by quizzes answered by parents and teachers (Conner's, Barkley, ADHD Rating Scales). There are three subtypes of ADHD: combined, inattention predominance and hyperactive-impulsive predominance. ADHD can be pure or can be presented with other pathologies of: learning, CT, depression, anxiety, Tourette, etc. . . . The lack of functionality is the most important factor in ADHD. Diagnosis is clinic; biological markers such as electroencephalogram, neuroimage studies or neuropsychological proofs are not needed and are unnecessary.

This disease is expressed by different complex forms of heritage with multiple gene factors in a lesser way plus ambient factors. Heritage is estimated as a 75% considering it as one of the neuropsychiatric diseases with the bigger heritage component. The principal hypothesis of the cause of ADHD is the dysfunction of the dopaminergic system. There are plenty evidence genetic and ambient ones in which there can be involved differences between cerebral anatomy and genotypic. Evidence suggests that ADHD is a polygenic disorder that involves at least 50 genes, because ADHD is usually present with other behavioral abnormalities.

Interviews and detection/gravity scales in which parents, patients and teachers generally give information about observed behaviors. Parents sometimes give valuable information about child and adolescent difficulties in homework, relations with brothers/sisters, friends. Teachers may give information of behavior in and out of the classroom, capacity of concentration, communications with classmates and the completion of school rules. The periodic recollection of this information by quizzes also help the physician to evaluate by an objective way the effect of the treatment in all functional areas of the patient. These scales, don't have the absolute power to give a definitive diagnosis, but are an excellent support implement.

Compositions according to this second embodiment show superior results in treating and/or preventing disorders of psychological development, such as in particular autism or Autistic Syndrome Disorder (ASD), apraxia, dyspraxia, or dyslexia.

Autism or Autistic syndrome disorder (ASD) is a developmental disorder that affects of early childhood between three and eight of every 10,000 school-aged children that affects brain function, interfering with reasoning ability, imagination, communication, and social interaction. The disease can include language disorders with impaired understanding, echolalia, pronominal reversal (such as using “you” instead of “I” or “me” when referring to one's self), rituals and compulsive phenomena, and uneven intellectual development with mental retardation. The cause of autism is unknown, but there are, at the least, some important genetic factors, as indicated by the fact the concordance rate is significantly greater in monozygotic twins than dizygotic twins. Merck Manual, 17^{th} edition, section 19, chapter 274 (1999); Autism Review, Lowell Ackerman, http://www.parentzone.com/autism/review.htm (1997). Other factors may include rubella, problems during pregnancy, labor and delivery, cytomegalic inclusion disease, phenylketonuria, and fragile X syndrome. Autistic children are also at increased risk of developing seizure disorders, especially during their teen years.

Apraxia is a central neurological speech disorder that affects the oral articulatory musculature. A child having this disorder is unfortunately unable to organize his or her oral muscles in order to produce speech sounds in isolation to sequence speech sounds (consonants and vowels in words), and finally to sequence the words into sentences.

Dyspraxia is recognized to be caused by an immaturity of brain development associated with poor synaptic transmission and possibly poor arborisation of neurons, that is to say a disorder with an organic basis. In practical terms dyspraxics are poorly coordinated, disorganized, have problems of ideation. Motor planning and execution so that written work and ball games are extremely difficult for them. Handwriting is poor. Poor memory, restlessness and impulsiveness may be features of the condition.

Dyslexia represents a condition of reduced ability to read and write, in the presence of adequate intelligence, conventional instruction and socio-cultural opportunity and without any ophthalmic abnormality. A proportion of those with this condition also display an inability to listen in the absence of any impairment in their hearing. The several manifestations of this condition have been encapsulated in the term ‘Dyslexia’, derived from the Greek ‘dys’—meaning difficult, and ‘lexis’—meaning words. A common expression describes ‘Dyslexia’ as ‘word blindness.

According to one embodiment, the present invention relates to a composition for use in a method of treating and/or preventing ADHD children and young adult comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereof;
c) γ-linolenic acid (GLA) and/or the pharmaceutically acceptable derivatives and/or precursors thereof, and
   in combination with standard care of ADHD, such as for example methylphenidate HCl or atomoxetine.

Most preferred synergistic compositions according to this embodiment comprise an increased concentration of EPA, DHA, and GLA. More particularly, a preferred ratio of EPA:DHA:GLA is equal to around 9:3:1. Thus the ratio may be around 9:2.5-3.5:0.8-1.2. In one embodiment the ratio is, or is around, 9:2.8-3.2:0.9-1.1. Also preferred standard care of ADHD of the combination is methylphenidate HCl (MPT).

The Applicant has showed in Examples 7 to 9 that the combination of omega 3/6 fatty acid in a dosage of EPA:DHA:GLA (9:3:1) with ADHD standard care significantly increased the efficacy and safety of the treatment in pediatric patients with ADHD. Indeed, as shown in the Examples, the combination substantially reduced both ADHD symptoms, i.e., inattention and hyperactivity. The safety of ADHD treatment was also improved since administration of EPA:DHA:GLA (9:3:1) in combination with ADHD standard care allowed to decrease the dose of ADHD standard care from 30 to 50% of the current ADHD treatment dosage. Furthermore, the Applicant demonstrated that use of the combination of EPA:DHA:GLA (in the ratio of around 9:3:1) allowed to substantially reduce and/or prevent adverse events that are generally observed when treating patients with ADHD standard care, such as weight reduction, hyporexia and anxiety.

Compositions of the invention are thus of particular benefit for treating and/or preventing behavioral and emotional disorders. Such disorders include hyperkinetic disorders, conduct disorders, disorders of social functioning, tic disorders, and feeding disorders. Hyperkinetic disorders are disturbance of activity and attention, such as Attention Deficit Hyperactivity Disorder (ADHD) or Attention Deficit Disorder (ADD), hyperactivity, or cognitive dysfunction. Behavioral and emotional disorders are disorders of psychological development and also include disorders of speech and language, specific developmental disorders of scholastic skills, specific developmental disorder of motor function, and pervasive developmental disorders. By way of examples, we may cite autism or Autistic Syndrome Disorder (ASD), apraxia, dyspraxia, or dyslexia. In one preferred embodiment the composition of the invention is used to prevent and/or treat patients who have ADD without hyperactivity. Such patients may have slow retrieval and information processing and/or low levels of alertness and/or problems with memory or orientation. The patients may have ‘sluggish cognitive temp (SCT)’.

According to one other aspect of the present invention, above-described compositions further comprise at least one antioxidant in addition to EPA, DHA, GLA, psychostimulant and/or anxiolytic compound, vitamins and minerals.

The term “antioxidant” as used herein means an agent that can prevent or reduce an oxidation, degradation and/or other decomposition that would otherwise occur to components or ingredients of the compositions of the invention, such as vitamins and/or minerals and/or edible oils. A wide variety of antioxidant agents are commercially available from sources known by those of skill in the art.

Compositions according to the present invention present improved stability measured in terms peroxide value and anisidine value and oxidation induction time. Lower stability of EFAs leads to decomposition reactions of fatty acids that form undesirable peroxides and hydroperoxides. The subsequent decomposition of these oxidation products can form volatile and non-volatile aldehydes and/or ketones. The non-volatile components can catalyze further oxidation of the oils and the volatile components give rise to undesirable taste and smell.

Such antioxidants may include for example at least one vitamin E isoform, tocopherols or any other agent that reduces oxidative stress. Preferred antioxidants are selected among tocopherols (4 toco), Origanox®, oils extracts from several spices, such as oregano (Origanum vulgare), thyme (Thymus vulgaris), rosemary (Rosmarinus officinalis) or rosemary extract, sage (Salvia officinalis), and clove (Syzygium aromaticum).

Origanox® which is commercialized by Harrington Nutritional, is a natural, completely water soluble, powerful antioxidant, extracted from edible herb species, belonging to the Labitae family (such as Origanum vulgare and Salvia officinalis) and considered as GRAS by the FDA. Origanox® is offered in various standardized grades of antioxidant activity.

Alternatively, such antioxidant may include vitamin C, synthetic antioxidants, e.g., BHT, TBHQ, ethoxyquin, alkyl gallates, hydroquinones, tocotrienols), NXY-059 (Disufenton sodium); chain-breaking phenolic antioxidants (such as Vitamin E and butylated hydroxytoluene [BHT]); phenyl-substituted nitrones; azulenyl-substituted nitrones; α-phenyl-N-tert-butyl nitrone; stilbazulenyl nitrone, polyphenolic plant extract with antioxidant properties etc. . . .

A preferred antioxidant for use according to the present invention is Origanox® OS-F commercialized by Frutarom. Origanox® OS-F is an oil dispersible extract from the edible herb specie Melissa Officinalis and Sunflower-Oil, dispersed with PGPR (Polyglycerol polyricinoleate) and acidified ascorbic acid. Another preferred antioxidant may be rosemary (Rosmarinus officinalis) or rosemary extract. Most preferably, the OXY'LESS® CLEAR commercialized by Naturex is used in the compositions of the present invention. OXY'LESS® CLEAR corresponds to an extract obtained from rosemary leaves or Rosmarinus officinalis L.

Minimizing the amount of oxidation measured by “Peroxide value” (PV) and “Anisidine value” (AV) can have significant implications when assessing the oxidative stability of oil. The present invention provides omega-3/6 fatty acid formulations in which the formulation comprises less than 20 meq/kg peroxides, less than 10 meq/kg peroxides, or less than 5 meq/kg peroxides. The present invention also provides omega-3/6 fatty acid formulations in which the formulation comprises less than 50 meq/kg anisidines, less than 30 meq/kg anisidines, around 25 meq/kg anisidines, or less than 15 meq/kg anisidines.

“Peroxide value” (PV) and “Anisidine value” (AV) as used herein are generally indices of deterioration in edible oil. The peroxide value (PV) is the concentration of peroxide compounds in the oil measured in meq/kg. Peroxide compounds are produced during PUFA oxidation, thus, the higher the value of PV, the more PUFA oxidation that has occurred. The AV indicates the amount of oxidation that the oil has experienced prior to measurement and is a measure of the concentration of the secondary oxidation products. The AV of oil is a measure of the amount of non-volatile aldehydes and/or ketones in the oil. As the AV of the oil measures the non-volatile aldehyde and/or ketone concentration in the oil (typically, unitless), it is a measure of its oxidative history. Aldehydes and ketones are produced from the decomposition of the peroxide or hydroperoxide species, which are primary oxidation products of the olefinic functionality on a fatty acid. Methods for measuring PV or AV of an oil are well known in the art.

Other preferred stabilized formulations are obtained by microencapsulation technique, which is a well known technique in the art.

According to one aspect, the present invention provides a food supplement comprising the foregoing compositions, and thus can be administered as a food supplement and/or a food supplement for special medical purpose. In another other aspect, the present invention provides a pharmaceutical composition comprising the foregoing composition; specifically said composition is in the form of a pharmaceutical composition, further comprising a pharmaceutically acceptable vehicle or carrier. In one embodiment administration is by in vivo generation of one or more of the specified substances/compositions of the invention. In this embodiment precursors of the substances are given to subject.

As used herein, “pharmaceutically acceptable carrier” or “pharmaceutically acceptable vehicle” includes any material which, when combined with an active ingredient of a composition, allows the ingredient to retain biological activity and without causing disruptive reactions with the subject's immune system. Examples include, but are not limited to, any of the standard pharmaceutical carriers such as a phosphate buffered saline solution, water, emulsions such as oil/water emulsion, and various types of oils and lipophilic solvents. Compositions comprising such carriers are formulated by well known conventional methods (see, for example, Remington's Pharmaceutical Sciences, Chapter 43, 14th Ed. or latest edition, Mack Publishing Co., Easton Pa. 18042, USA; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. Typically any of the substances mentioned herein are administered in isolated or purified form. In one embodiment the stimulant or anxiolytic is in a ‘short-acting’ form, and is not in the form of a slow release (retard) formulation. In the case of methylphenidate this may be in methylphenidate galenic form.

Both synergistic stabilized food supplement and synergistic stabilized pharmaceutical compositions according to the present invention are preferably in a form suitable for oral consumption.

By way of example, oral synergistic stable food supplement or pharmaceutical compositions may be in the form of hard or soft capsule, microcapsule, oil, syrup, suspension, solid or semi-solid, tablet, sugar-coated tablets, powder, pellet, film-coated tablet, granule, emulsion, paste, gels, lozenges, gums, or chewable formulation, such as chewing-gum. These oral formulations are preferably gastro-resistant formulations.

Most preferably, such compositions are in the form either of hard capsules of gelatin, or soft gel capsule which present a one-piece, hermetically sealed soft gelatin shell that is filled with a material, substance or compositions (a “fill”). The soft gel shell is generally comprised of a film-forming material, such as gelatin, for example type A and/or type B, and water-dispersible or water-soluble plasticizers (to impart flexibility). Soft gel capsules may be produced using known methods and conventional machinery. The size of the capsules is preferably so chosen that the preferred daily dose is provided by 1, 2, 3, 6 or 9 capsules.

The present composition may also include other pharmaceutically acceptable carriers and desirable additives such as, without limitation, starches, sugars, fats, amino acids, proteins, derivatives thereof or combinations thereof. Inclusions of additives which assist in formulating the final composition are also desirable. In one embodiment the administered composition comprises as active ingredients only the specified substances and no other substances. In another embodiment the composition which is administered comprises only 4, 5 or 6 to 10 active ingredients.

Alternatively, the compositions of the present invention combination may be administered as an oil to be taken in liquid form or as an oily suspension, for example added to normal food. Preferably such oil contains further fat-soluble flavors in order to change or mask the fishy taste of the combination. When administered as syrup the combination is provided as a emulsion in water containing emulsifying compounds and suitable flavors. Oils, oily suspensions or syrups are preferably provided in containers allowing simple dosing.

Pharmaceutical or food supplement dosage forms may contain excipients. Excipients include fillers, dyes or pigments, flavors, stabilizers, extenders, binders, humidifiers, surfactants, lubricants, and the like. Excipients must be of sufficiently high purity and sufficiently low toxicity to render them suitable for administration to the subject being treated. An excipient can be inert or it can possess pharmaceutical benefits. Excipients are selected with respect to the intended form of administration, e.g. oral tablets, capsules, powders, syrups, emulsions, suspensions, and the like, and consistent with conventional pharmaceutical practices. For example, for oral administration in the form of gel capsule the omega-3 fatty acid formulation may be combined with filler, a preservative, flavorant, colorant or the like.

Stabilizers may be added to improve the stability of EFAs. While the relationship of the concentration of stabilizers to the rate of fatty acid oxidation depends on the specific stabilizer, this relationship can be complicated by the presence of more than one stabilizer. The addition of multiple stabilizers can act to stabilize each other and when this occurs, a combination of two or more stabilizers can be more effective at terminating free radicals than a single stabilizer or at the same time the different stabilizers may antagonize each other's activities. Hence optimizing the right stabilizer combinations to improve stability of the EFA formulation is not a matter of routine experimentation. The present inventors have invented the correct combination of elements which makes the formulations of the invention unique in their combination and stability.

The present composition is advantageously formulated into a convenient dosage form, such as a tablet or a film coated tablet, or enclosed in a shell or capsule. Also included herein are pharmaceutical compositions, comprising pharmaceutical formulations in a unit dosage form. In such dosage forms, the formulation is subdivided into suitably sized unit doses containing appropriate quantities of the omega-3 fatty acids, an effective amount to achieve the desired purpose. Formulation of the desired ingredients, nutrients and/or additives may be accomplished by conventional methods. Advantageously, the final composition is encapsulated in a capsule, such as a gelatin capsule, and more advantageously, a soft-shell gelatin capsule. For example, compositions including the vitamins, minerals, EPA DHA and GLA in desired forms and quantities, can be made by weighing the individual ingredients, and blending them together to form a homogeneous mixture for final formulation or encapsulation. Other oral formulations within the scope of the invention includes microcapsule, suspension, solid or semi-solid, tablet, sugar-coated tablets, powder, pellet, film-coated tablet, granule, emulsion, paste, gels, chewable formulation, gastro-resistant formulation, extended release formulations, sustained release formulations, extended release formulations, intermediate release formulations, oral formulations with phase release, like monophasic, biphasic release and the like.

Packaged pharmaceutical formulations are included herein. The invention includes providing prescribing information, over the counter medical use information, or nutritional information for the dosage form, for example, to a patient or health care provider, or as a label in a packaged pharmaceutical formulation. Information included in the pharmaceutical package may include for example efficacy, dosage and administration, contraindication and adverse reaction information pertaining to the dosage form. The formulations of the present invention may also be provided in combination with vitamins or mineral supplements either in a single unit dosage form or in separate unit dosage forms. In one embodiment the invention provides a method of preventing or treating a subject comprising first carrying out a diagnostic test for any of the conditions mentioned herein and if the subject is found to be at risk of the condition or if the subject has the condition, administering to the subject the composition of the invention.

The different components of the composition of the invention can be administered as part of the same composition or separately to the subject. Thus the invention provides a product comprising a) Docosahexaenoic acid (DHA) and/or the pharmaceutically acceptable derivatives and/or precursors thereof; b) Eicosapentaenoic acid (EPA) and/or the pharmaceutically acceptable derivatives and/or precursors thereof; c) γ-linolenic acid (GLA) and/or the pharmaceutically acceptable derivatives and/or precursors thereof; and at least a psychostimulant compound selected among methylphenidate HCl, atomoxetine, amphetamine, gamma-hydroxybutyrate, dextroamphetamine, sibutramine, methylenedioxymethamphetamine, and/or an anxiolytic compound selected among fluoxatine, sertraline, paroxetine, fluoroxamine, citralopram, venlafaxine, bupropion, nefazodone, and mirtazapine; for simultaneous, separate or sequential administration.

### EXAMPLES

### Example 1

### Example 1.1

Different admixtures of EPA, DHA and GLA (relative concentration of EPA:DHA:GLA 9:3:1), combined with different salts of iron and zinc and with calcium pantothenate were assayed by standard pharmacopoeial methods (PhEUR, peroxides as primary oxidation products (PV)). Different Fe (II) salts and a Fe (III)-hydroxy-polymaltose complex (Maltofer®), inorganic and organic Zn salts and Ca-panthotenate were used in half RDA-related concentrations. The various mixtures were poured in brown snap cap vials and tested after 1 week at room temperature for the peroxide value (PV). For the analysis the samples were centrifuged at 4000 rpm for 10 minutes and the oxidation parameters were analyzed in the supernatant.

### Sample 1

| | | |
|---|---|---|
| Fish oil | 560 | mg |
| EPO | 100 | mg |
| Iron fumarate | 21.3 | mg |
| Zinc oxide | 6.2 | mg |
| Calcium pantothenate | 6.5 | mg |

### Sample 2

| | | |
|---|---|---|
| Fish oil | 560 | mg |
| EPO | 100 | mg |
| Iron fumarate | 21.3 | mg |
| Zinc gluconate | 34.9 | mg |
| Calcium pantothenate | 6.5 | mg |

### Sample 3

| | | |
|---|---|---|
| Fish oil | 560 | mg |
| EPO | 100 | mg |
| Iron fumarate | 21.3 | mg |
| Zinc sulfate | 13.7 | mg |
| Calcium pantothenate | 6.5 | mg |

### Sample 4

| | | |
|---|---|---|
| Fish oil | 560 | mg |
| EPO | 100 | mg |
| Iron sulfate | 34.9 | mg |
| Zinc oxide | 6.2 | mg |
| Calcium pantothenate | 6.5 | mg |

### Sample 5

| | | |
|---|---|---|
| Fish oil | 560 | mg |
| EPO | 100 | mg |
| Iron sulfate | 34.8 | mg |
| Zinc gluconate | 34.9 | mg |
| Calcium pantothenate | 6.5 | mg |

### Sample 6

| | | |
|---|---|---|
| Fish oil | 560 | mg |
| EPO | 100 | mg |
| Iron sulfate | 34.8 | mg |
| Zinc sulfate | 13.7 | mg |
| Calcium pantothenate | 6.5 | mg |

### Sample 7

| | | |
|---|---|---|
| Fish oil | 560 | mg |
| EPO | 100 | mg |
| Maltofer | 26.2 | mg |
| Zinc oxide | 6.2 | mg |
| Calcium pantothenate | 6.5 | mg |

### Sample 8

| | | |
|---|---|---|
| Fish oil | 560 | mg |
| EPO | 100 | mg |
| Maltofer | 26.2 | mg |
| Zinc gluconate | 34.9 | mg |
| Calcium pantothenate | 6.5 | mg |

### Sample 8

| | | |
|---|---|---|
| Fish oil | 560 | mg |
| EPO | 100 | mg |
| Maltofer | 26.2 | mg |
| Zinc sulfate | 13.7 | mg |
| Calcium pantothenate | 6.5 | mg |

| | PV T = 0 day | PV T = 7 days |
|---|---|---|
| Sample 1 | 2.6 | 10.2 |
| Sample 2 | 2.3 | 3.8 |
| Sample 3 | 2.0 | 5.1 |
| Sample 4 | 0.6 | 2.2 |
| Sample 5 | 0.4 | 1.1 |
| Sample 6 | 0.5 | 2.3 |
| Sample 7 | 0.5 | 2.9 |
| Sample 8 | 0.5 | 2.7 |
| Sample 9 | 0.3 | 3.7 |

### Example 1.2

### Stability Assessment

A tocopherol-stabilized triglyceride admixture of EPA, DHA, and GLA (relative concentration of EPA: DHA: GLA (γ-linoleic acid; ω-6 PUFA)=9:3:1) was assayed by standard pharmacopoeial stability tests (Ph. Eur. peroxides as primary oxidation products with peroxide value [PV]), secondary oxidation products by the p-anisidine test [AV]). An oily suspension with the minerals and Vitamin B5 was tested in brown snap cap vials. Different Fe (II) salts and a Fe (III)-hydroxy-polymaltose complex (Maltofer®), inorganic and organic Zn salts and Ca-panthotenate were used in RDA-related concentrations. Samples were stressed at elevated temp. [40±2° C. and 75±2% relative humidity] over 4 weeks. For the analysis the samples were centrifuged at 4000 rpm for 10 minutes and the oxidation parameters were analyzed in the supernatant. A plant-derived antioxidant (Origanox OS-F®) was checked at 40° C. for improving stability.

### Results

[mean±SD]: as a blank saturated medium chain triglycerides (Miglyol®) were used;

| | Mygliol ® + minerals | Equazen ® | +Fe sulfate (FS) +Fe fumarate (FF) +Maltofer ® (MF) | +Zn sulfate (ZS) +Zn lactate (ZL) | $\begin{matrix}( \begin{matrix}+ MF \\ + ZL\end{matrix} ) \\ + Ca pantothenate \\ + Organox OS \text{-} F ®\end{matrix}$ |
|---|---|---|---|---|---|
| PV (d0) | 0 | 1.03 ± 0.05 | 2.79 ± 0.01 | 4.98 ± 0.01 | 1.20 ± 0.01 |
| (d 6) | | | 3.36 ± 0.04 | 1.53 ± 0.05 | 0.0 ± 0.0 |
| (d28) | | | 2.86 ± 0.04 | | |
| (n = 3) | 0 | 14.16 ± 0.02* | 4.23 ± 0.05 | 9.65 ± 0.04 | 12.59 ± 0.28* |
| | | | 13.13 ± 0.04* | 13.17 ± 0.07* | 1.30 ± 0.01 |
| | | | 11.78 ± 0.10* | | |
| | 0 | 38.66 ± 0.22* | 18.20 ± 0.00* | 18.97 ± 0.25* | 20.75 ± 0.14* |
| | | | 25.60 ± 0.00* | 10.97 ± 0.02* | 16.32 ± 0.09* |
| | | | 27.50 ± 0.00* | | |
| AV (d = 0) | 0 | 6.89 ± 0.88 | 8.35 ± 0.73 | 10.17 ± 0.10 | 2.09 ± 0.32 |
| (d28) | | | 8.37 ± 0.42 | 6.27 (n = 1) | 6.11 ± 0.08 |
| (n = 2) | | | 7.44 ± 0.39 | | |
| | 0 | 24.13 ± 2.52 | 143.33 ± 2.04* | 139.58 ± 4.74* | 49.75 ± 0.20* |
| | | | 329.9 (n = 1)* | 31.28 ± 1.04* | 14.13 ± 0.38 |
| | | | 150.42 ± 0.04* | | |

*exceeding limits

### Conclusions:

In contact with ambient air (oxygen) and at elevated temperatures the fatty acids formulations are readily oxidized despite the tocopherol presence (exceeding Ph. Eur. limits for PV (<10) and/or AV (<30) within 4 weeks).
Primary peroxides are differently transformed into secondary products. This process is catalyzed by minerals (Fe, Zn), in a formulation-dependent manner: Fe (III)<Fe (II); organic Zn lactate<inorganic Zn sulfate. Incorporation of a plant-derived antioxidant increased the stability significantly (4 weeks stress test within the limits).
A composition testing of destabilizing components in simple liquid formulations allows a rational development of a combined fatty acids nutraceutical and to eliminate inappropriate components.

### Example 2

### Example 2.1

### Preclinical Study

Knock out (KO) rats being deficient for the dopamine transporter (DAT) are used for assessing the impacts of the composition according to the present invention. Tested KO rats are young animals, e.g., around 3-month old with symptoms of hyperactivity or ADHD similar to junior subjects. (Spielewoy C, Roubert C, Hamon M, Nosten-Bertrand M, Betancur C, Giros B. Behavioural disturbances associated with hyperdopaminergia in dopamine-transporter knockout mice. Behav Pharmacol. 2000 June; 11 (3-4):279-90).

DAT-KO rats are divided in several groups which receive (1) the mixture of EPA, DHA, and GLA; (2) the B5, iron and zinc sources; (3) the mixture of EPA, DHA, GLA, vitamin B5, zinc, and iron. A placebo controlled group of KO 3-month old rat is also used as negative control.

Both treated and placebo groups are assessed by the following experiments: (i) behavioral experiments, and (ii) biochemical experiments, e.g., measure of the status of fatty acid in membranes of erythrocytes and neurons.

### Example 2.2

### Treatment of Junior Subjects with ADHD

The aim of this study is to investigate the effects of the composition according to the present invention comprising the mixture of EFAs (EPA, DHA, and GLA) with vitamin B5, zinc and iron on the treatment and/or prevention of ADHD behaviour, cognition and brain function. The study population of children with the clinical diagnosis of ADHD combined type, including co-morbidity, between 6 and 15 years, is divided in separated groups which are administered (1) EFAs; (2) EFAs+vitamin B5, zinc and iron; (3) placebo group receiving a capsule that has similar taste.

It is thought that the combination of the present invention improves ADHD and oppositional behaviours, neuropsychological performance on disorder-relevant cognitive tasks and improves brain dysfunctions in fMRI.

Subjects are tested before and after intervention on following parameters:
Behaviour
ADHD symptoms (Conner's)
Social and antisocial traits
Mood, depression and anxiety
Neuropsychological performance
Various fMRI tasks

### Example 3

Young wild type rats (3 month old) are used as an in vivo model for assessing the impacts of the composition according to the present invention on mainstream junior subjects.

Tested rats are divided in several groups which receive (1) the mixture of EPA, DHA, and GLA; (2) the vitamin B5, iron and zinc sources; (3) the compositions according to the present invention comprising the mixture of EPA, DHA, GLA, vitamin B5, zinc, and iron. A placebo controlled group of 3 month old rat is also used as negative control.

Both treated and placebo groups are assessed by the following experiments: (i) behavioral experiments, and (ii) biochemical experiments, e.g., measure of the status of fatty acid in membranes of erythrocytes and neurons.

### Example 4

Knock out (KO) rats being deficient for the dopamine transporter (DAT) are used for assessing the impacts of the composition according to the present invention. Tested KO rats are also 1-2 years old and present the symptoms of hyperactivity similar to hyperactive adult subjects. DAT-KO rats are divided in several groups which receive (1) the mixture of EPA, DHA, and GLA; (2) vitamin B5 and optionally other vitamins and minerals; (3) the mixture of EPA, DHA, GLA, and vitamin B5 and optionally other vitamins and minerals. A placebo controlled group of KO rat of the same age is also used as negative control.

Both treated and placebo groups are assessed by the following experiments: (i) behavioral experiments, and (ii) biochemical experiments, e.g., measure of the status of fatty acid in membranes of erythrocytes and neurons.

### Example 5

### Effects of the Composition in Combination with a Stimulant or Psychostimulant on ADHD Children

The study aims to evaluate the efficacy and security of the use of the compositions of the present invention in combination with methylphenidate for the treatment and/or the prevention of symptoms of ADHD in a pediatric population.

In particular, the study allows to compare (i) the clinical response measured by the Conner's score and DSM-IV criteria for ADHD, (ii) the cognitive interference effect, and (iii) the clinical response with the percentage of activation in the dorsal anterior cingular cortex before and after treatment in the group treated with methylphenidate with the group receiving the composition of the invention and finally with a group receiving combination of both treatment.

The study population is divided into the following groups A to C:
Group A receives methylphenidate with an initial dose of 0.5 mg/kg/day with weekly increase until achieve a dose of 1 mg/kg/d two doses administrated at 8 and 12 pm;
Group B receives the composition of the present invention at a dose of 7 g/day divided in two doses, daily administration morning and night; and
Group C receives methylphenidate with an initial dose of 0.5 mg/kg/day with weekly increase until achieve a doses of 1 mg/kg/day two doses administrated at 8 and 12 pm and the composition according to the present invention at a dose of 7 g/day divided in two doses, daily administration morning and night.

A clinical evaluation of each group is then conducted to assess the following clinical effects at one-month, three-month, six-month and a year follow up:
DSM-IVR (Statistical Manual of Mental Disorders fourth edition) criterion to reach the diagnosis of ADHD;
ADHD rating scale;
WISC-IV test to evaluate the Verbal Comprehension Index (VCI), Perceptual Reasoning Index (PRI), Working Memory Index (WMI), and Processing Speed Index (PSI);
Conner's test;
Bender and Rey's figure for neuropsychological maturity.

### Example 6

### Efficacy and Safety of Omega-3/6 Fatty Acid Supplementation Alone or in Combination with Methylphenidate in Pediatric Patients with ADHD

### Objectives

ADHD is one of the most frequent disorders that affect children and produce scholar failure and behavioral problems; prevalence is 7% in Latin America. The study was initiated to investigate the effects of omega 3/6 in Latin-American children diagnosed with ADHD, alone and in combination with methylphenidate (MTP).

### Methods

Newly diagnosed ADHD children of both genders, 6 to 12 years old, were included in a RCT with one year follow-up in a 3-arm design (A: Omega-3/6; B: Omega 3/6+MTP; and C: MTP). The omega-3/6 fatty acid dosage used was EPA/DHA/GLA (9:3:1) in a total of 792 mg/day (Equazen™). MTP was prescribed in a dosage of 1 mg/kg/day (titration).

The ADHD diagnosis and follow up were made using the Diagnostic and Statistical Manual of Mental Disorders fourth edition (DSM-IV) scale, the ADHD Rating Scale Spanish version (ADHD-RS-VE), and the CEAL (Latin-American scale for ADHD) and overall clinical impression (CGI). For clinically meaningful response, a reduction of at least 25% of the symptoms was used. The adverse events (AE) were evaluated by the parents based on the impact on the patients.

### Results

30 patients by group were recruited; the combined subtype was the most frequent (56%). 85% finished the one year follow-up; with 75% in all three groups reporting a 50% or more reduction in symptoms. While no difference was observed between groups regarding the effect on inattention, omega-3/6 supplementation seemed to have less effect on hyperactivity. The most frequent AE were weight reduction, hyporexia, and anxiety (MPT group). In the combined group (Omega-3/6 plus MTP), hyporexia and weight reduction were significantly lower.

### Conclusions

Nutritional supplementation with omega-3/6 fatty acids was effective and safe and can be considered as a good option as monotherapy in inattentive ADHD children and as an adjunctive therapy in combined type, helping not only to improve the symptoms but to lower side effects of MPT.

### Example 7

### Further Study on Efficacy and Safety of Omega-3/Omega-6 Combination with Methylphenidate

### Study Design

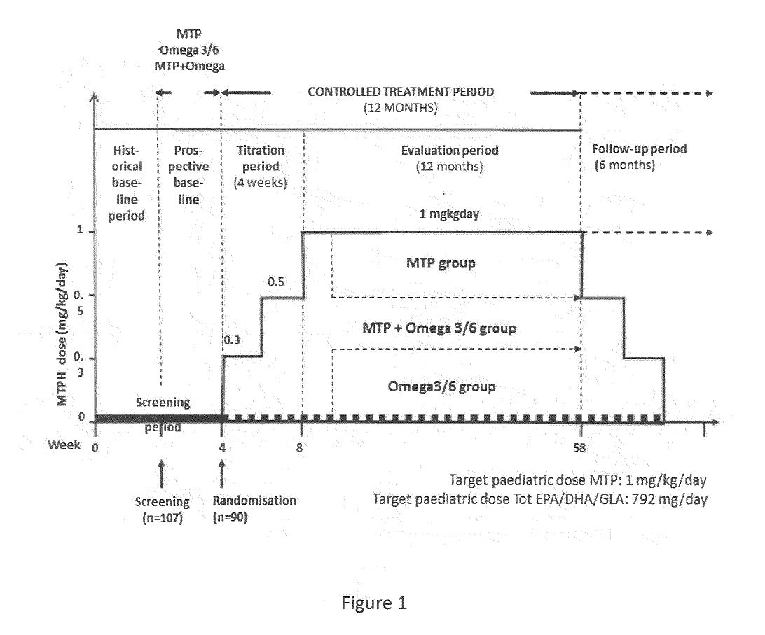

The trial was performed at the Neurology Department of the Hospital Infantil de México Federico Gomez (Health National Institute for children's in Mexico) as a randomized pilot trial. Three treatment groups were compared: group A: MTP; group B: Omega 3/6; group C: MTP+Omega 3/6. Subjects were randomized to one of these groups after screening. After inclusion to the trial, the subjects underwent three different trial periods: the 4 weeks titration period (only for the group A and group C in relation to the MTP), the 12 months evaluation period and the 6 months follow up period (FIG. 1)

### Informed Consent and Ethics

Written informed consent to participate was given by the parents or tutors. The trial was approved by the local ethical review board.

### Study Subjects

A total of 107 children between 6 and 12 years of age with a newly diagnosed ADHD entered the screening phase of the trial. The presence of any previous ADHD treatment was an exclusion criterion. 17 children were screening failures and could not be included to the trial. After written informed consent by the parents or tutors of the children, a total of 90 patients were distributed to the three groups following a randomization to the three groups against a set of arbitrary numbers.

Inclusion criteria: Patients from 6 to 12 years old, diagnosed as having attention-deficit hyperactivity disorder by the DSM-IV, agreement to take part in the survey (letter of informed consent signed by the parent or tutor and letter of acknowledgement). Exclusion criteria: any Axis I psychiatric diagnosis other than ADHD except oppositional defiant behaviour disorder, neurological deficit, seizures or tics; previous treatment of suitable doses of methylphenidate; patients with known hypersensitivity to omega-3. Criteria for withdrawal: not attending the monthly follow-up, non-compliance to the treatment (performed with capsule count), not completing the control assessments within 12 weeks, patients who display hypersensitivity to the omega-3/6 complex or to MTP, patients who after the three-month assessments showed no adequate response to the therapy or display adverse effects that prevented them from continuing with the treatment, worsening on the clinical follow-up scale that required being withdrawn from the programme and being administered a different therapy.

### Study Treatments

The omega-3/omega6 fatty acid supplement was used. The daily dosage of this omega 3/6 was of three capsules twice daily, corresponding to a daily dose of 558 mg EPA, 174 mg DHA and 60 mg gamma-linolenic. This specific product was chosen because it was the product and the daily dosage used in three previous clinical studies showing significant positive improvements on ADHD related symptoms without showing significant adverse events.

The stimulant used in this trial was short or long acting methylphenidate (hereafter termed MTP). The daily dose was calculated to start at a concentration of 0.5 mg/kg/day to reach a maximum of 1 mg/kg/day depending on the response and the tolerance. The doses were adjusted weekly.

### Measures and Procedures

Clinical assessments were made at four time points: at baseline, after 3 months, after 6 months and after 12 months. The study consisted in six stages with a total of 12 visits including the screening visit (screening visit 0) and the period of selection and titration of the medication in the case of the group A & C (first 4 weeks). At baseline all the included patients were newly diagnosed with ADHD being drug-naïve (no previous treatment with omega 3/6, MTP or atomoxetine was permitted). ADHD diagnosis was made for patients who met DMS-IV criteria of any subtype and by using the ADHD-RS-VE (Spanish version) and the CEAL (Latin American scale for ADHD). The presence of any associated condition (comorbidity) was assessed by clinical interview according to DSM-IV criteria. The investigator made a medical evaluation, including weight, height, cephalic perimeter, neurological evaluation, sleep patrons and other comorbidities. A questionnaire for the symptoms of fatty acids deficiency was applied, where 0 means without symptoms and 3 with a high degree of symptoms. We analyzed blood samples for biometry hematic and hepatic function.

For clinically meaningful response, definition of clinical trials with ADHD medications was used, that is: a reduction of at least 25% of the symptoms scores on the ADHD-RS scale. The other important variable was the permanence time in the study measuring efficacy and tolerability. The overall clinical impression of symptom severity and functional impairment was measured with the CGI severity scale, which is a clinician rating of the patient's symptom severity related to the clinician's total experience with ADHD patients. The CGI goes from 1 (normal, not ill) to 7 (among the most extremely ill patients). This was applied to the parents too, from 1 (normal, as other kids) to 7 (the worst children).

The patients was evaluated at baseline including a physical examination, including height and weight, pulse and blood pressure, a neuromotor examination and assessment of the general level of functioning with CGI. We evaluated each week for the first four weeks and each month for a year. The adverse events (AE) were evaluated at any clinic event appointed by the parents and with an impact over the patients, independently if it was associated or not with the medication. This includes exacerbation of the preexistence symptoms. It was important to note the moment of these AE. If these adverse effects did not disappear or were persistent, we proposed to modify the treatment.

Once the parents signed the informed consent, patients were alleatorized in three different groups: MTP (Group A), Omega 3/6 (Group B) and MTP+Omega 3/6 (Group C). All the parents were capacitated for the administration of the medication and the methods to count the capsules. Pill counting for protocol compliance was performed at each visit considering has a good medication if at least the 85% of the doses was ingested.

### Results

### Demographic Variables

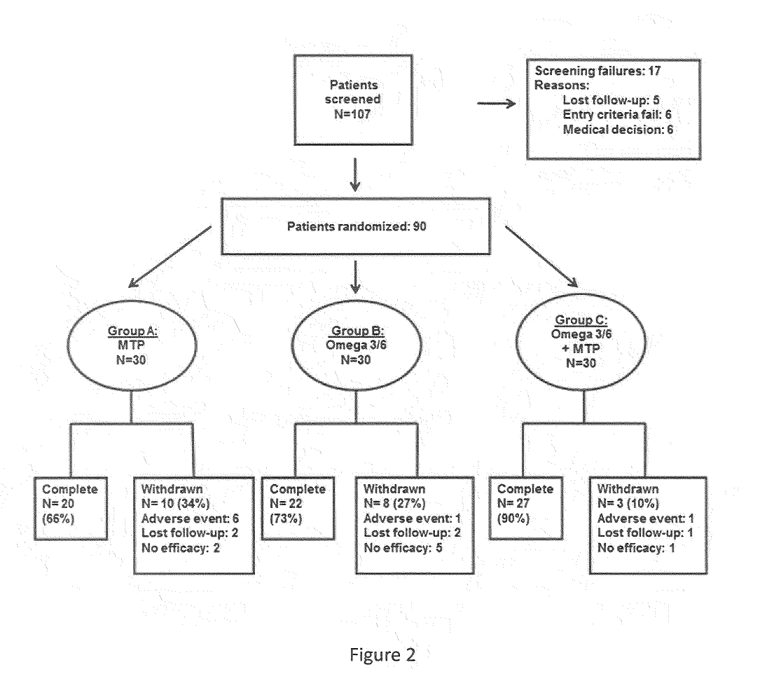

One hundred and seven patients were screened to receive treatment; only 90 patients completed all criteria and were randomized to participate (n=30 patients/group). Patient's characteristics and diagnosis are summarized in table 1. Patient disposition to group A, B or C is summarized in FIG. 2. Baseline symptom severity and symptom change during treatment are summarized in table 2. At baseline, the three groups did not differ significantly from each other in all measures. Mean baseline severity for ADHD suggested moderate to severe symptoms. In each of the three groups of treatment there was a predominance of males (A: 20; B: 22; C: 19 respectively), and the more frequent subtype was the combined group (57%) following for sluggish (23%), Inattentive (12%) and hyperactivity (8%). The more frequently present comorbidities were learning disorders, conduct disorders and anxiety. The highest rates between the scales were for the combined group.

### 3.2. Reasons of Withdrawn and MTP Doses

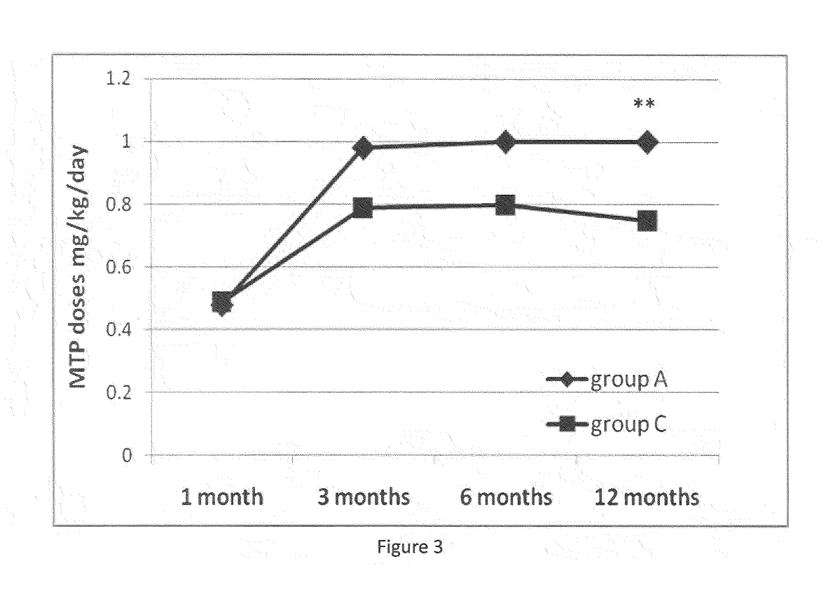

The continuity over the study was different between groups. 66% of the patients in group A, 73% of the patients in group B and 90% of the patients in group C completed the treatment period of 12 months. The causes for these withdrawn were different between the groups. In group A 6 patients left the study because of side effects, 2 due to loss of follow-up and 2 for lack of efficacy; in group B one patient withdraw for side effects, 2 due to loss of follow-up and five for lack of efficacy and in group C 3 patients withdraw (1 patient per each reason above) (FIG. 2). The starting dose for MTP (for the groups A and C) was 0.5 mg/kg/day. The titration and the increment of the doses were made under the best clinical estimated conditions to adjust the MTP doses to a maximum of 1 mg/kg/day. The MTP doses were adjusted individually showing a significant difference between the final MTP dose after 12 months for group A and group C (A: 1.0 mg/kg/day vs. C, 0.75 mg/kg/day, p<0.001, FIG. 3).

The doses of Omega3/6 were not modified during the whole clinical trial.

### Side Effects

No serious adverse event was reported during this clinical trial. The side effects encountered are showed in table 2. The most frequent side effect was hyporexia encountered in 16 patients of the group A (53.3%) and in 8 patients of the group C (26.7%). Only 2 patients (6.7%) of the group B reported hyporexia as side effect. Side effects like headache, irritability, tension, pallor, palpitation sleeplessness, tics, tremor and seasickness were only reported in the group A and C, the both groups containing MTP.

In group B the most frequent reported side effect was dyspepsia, encountered by 9 (30.0%) patients after one month of treatment, followed by diarrhea reported by 5 patients (16.7%) after the same period. In mostly all the cases the dyspepsia and the diarrhea disappeared rapidly after the first treatment month.

### ADHD Rating Scales (ADHD-RS-SV) and Clinical Global Impression (CGI)

The ADHD-RS-SV (total and subtypes) and the CGI were measured at baseline, after one, three, six and 12 months treatment.

In all three groups significant improvements in the ADHD-RS-SV Total (parents) and the CGI (parents and investigators) were measured already after three months of treatment.

### ADHD Rating Scales (Parent ADHD-RS-SV)

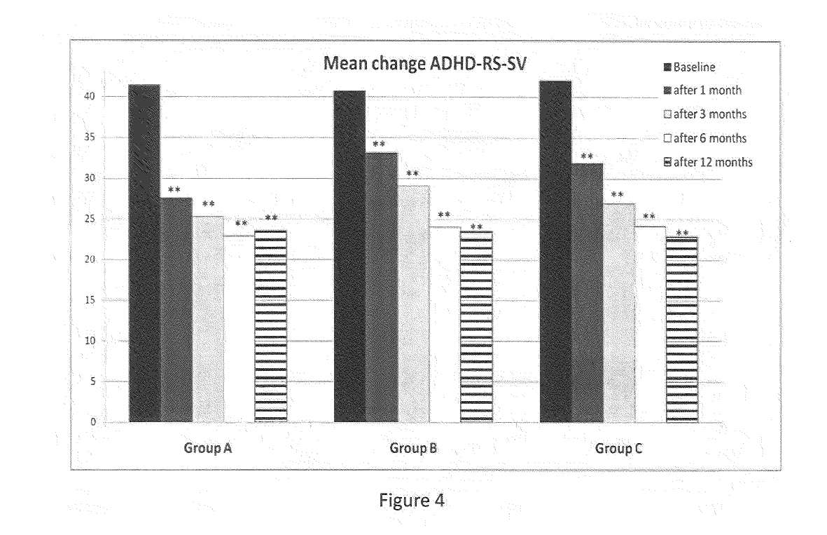

The fastest significant decrease in the ADHD RS total scores was seen in group A after one month treatment, with a score of 41.43±4.32 at baseline compared to 27.6±4.97) after one month (p<0.001). After 8 weeks, however, a difference between the groups was not observed any more. In group B and group C a 25% improvement of the ADHD symptoms occurred at three months (Group B: 40.7±4.82 vs. 29.07±7.15, p<0.001; Group C, 42.03±4.00 vs. 26.96±4.87, p<0.001; FIG. 4). A further significant decrease of the ADHD RS total scores was observed in all three groups after 12 months as depicted in FIG. 4.

As for the ADHD-RS-SV total scores also for the inattentive and the hyperactive/impulsive subscales a statistically significant decrease was observed in all three groups. The inattentive symptoms decreased on a comparable manner over time for all three groups decreasing from a score of 21.47±3.20 to a score of 12.04±1.78 after three and to 11.45±1.32 after 12 months for group A (p<0.001), from a score of 21.47±3.20 to a score of 12.96±2.75 after three and to 10.95±1.00 after 12 months for group B (p<0.001) and from a score of 21.57±2.84 to a score of 12.43±2.04 after three and to 10.78±0.85 after 12 months for group C (p<0.001). No significant difference were encountered between the inattentive score decreases for the different treatment groups (Table 3)

The strongest decrease of the hyperactivity/impulsivity symptoms were observed in group A (baseline: 19.93±5.11, after 3 months: 13.25±3.05 and after 12 months: 12.1±1.29; p<0.001), followed by group C (baseline: 20.6±5.45, after 3 months: 14.46±3.12 and after 12 months: 12.11±1.42; p<0.001) and finally by group B (baseline: 19.37±6.06, after 3 months: 16.11±5.41 and after 12 months: 12.55±2.24; p<0.001). The difference in the amount of decrease in the hyperactivity/impulsivity subscales was not statistically different between the three groups (table 3).

### Clinical Global Impression (CGI)

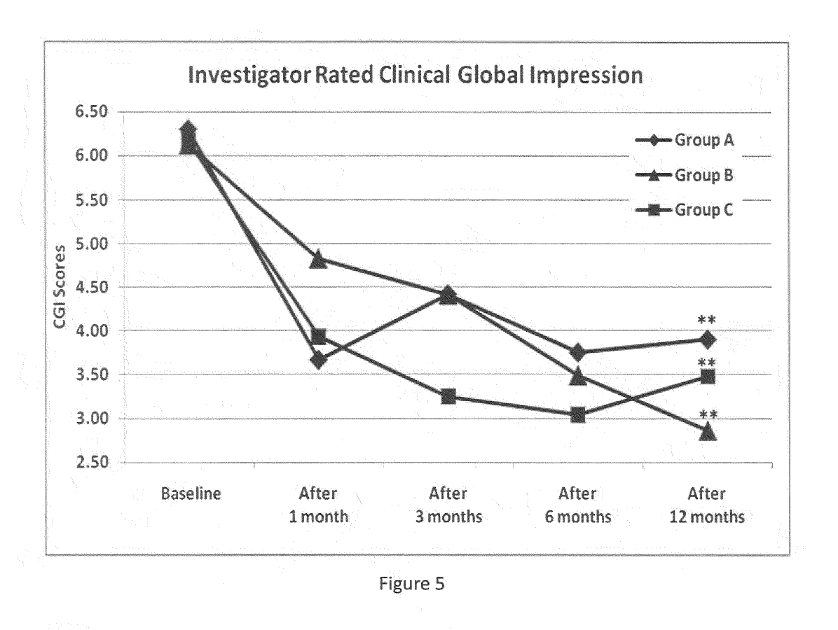

At baseline the investigator rated CGI was comparable for all three groups (p>0.05). With baselines means of 6.30±0.65 for group A, 6.13±0.63 for group B and 6.17±0.63 for group C, the patients presented a marked to severe CGI severity. A statistically significant improvement of the CGI score was observed in every group already after the first months, with the strongest CGI score improvements for the groups A and C at this measurement time. After a strong and fast decrease of the CGI scores in the first treatment month (3.67±1.21, p<0.001) the group A showed again a slight increase of the CGI severity to 4.42±1.02 at three, 3.75±0.44 at six and 3.90±0.45 at 6 months. The strong and fast increase was also observed in group C, where the CGI severity continued to decrease until month 6 with a slight increase at 12 months (at 3 months: 3.25±0.75, at six month: 3.04±0.44 and at 12 month: 3.48±0.85; p<0.001). In contrast to these two groups, group B showed a constant and statistically significant decrease of the CGI severity throughout the entire treatment period with values of 4.41±1.12 at three months, 3.49±0.67 at six month and 2.86±0.56 at 12 month (p<0.001). The CGI severity decreased from marked/severe before the treatment to mild/moderate for group A and C and to borderline/mild for group B (FIG. 5).

The results of the parents rated CGI severity were comparable to the investigator rated ones (data not shown).

### Clinical Global Impression (CGI)

At baseline the investigator rated CGI was comparable for all three groups (p>0.05). With baselines means of 6.30±0.65 for group A, 6.13±0.63 for group B and 6.17±0.63 for group C, the patients presented a marked to severe CGI severity. A statistically significant improvement of the CGI score was observed in every group already after the first months, with the strongest CGI score improvements for the groups A and C at this measurement time. After a strong and fast decrease of the CGI scores in the first treatment month (3.67±1.21, p<0.001) the group A showed again a slight increase of the CGI severity to 4.42±1.02 at three, 3.75±0.44 at six and 3.90±0.45 at 6 months. The strong and fast increase was also observed in group C, where the CGI severity continued to decrease until month 6 with a slight increase at 12 months (at 3 months: 3.25±0.75, at six month: 3.04±0.44 and at 12 month: 3.48±0.85; p<0.001). In contrast to these two groups, group B showed a constant and statistically significant decrease of the CGI severity throughout the entire treatment period with values of 4.41±1.12 at three months, 3.49±0.67 at six month and 2.86±0.56 at 12 month (p<0.001). The CGI severity decreased from marked/severe before the treatment to mild/moderate for group A and C and to borderline/mild for group B (FIG. 5).

The results of the parents rated CGI severity were comparable to the investigator rated ones (data not shown).

### Discussion

Attention Deficit Hyperactivity Disorder (ADHD) is a frequent complaint that has an impact on school performance levels and the family and social ties of the patients suffering from it. Treatment of ADHD is multimodal and the cornerstone of its treatment is considered to be pharmacological. However, in spite of the efficacy reported on drugs, such as stimulants (methylphenidate) and non-stimulants (atomoxetine), poor effectiveness in some cases, reported side-effects, and concern over long-term effects, have led to the need for exploring other alternative treatments that offer better options for treating these patients.

There are studies which show evidence of a possible polyunsaturated fatty acid deficiency in patients with ADHD and other neurodevelopment disorders, such as dyspraxia, dyslexia and autism. These studies have reported improvement in ADHD symptoms with the use of polyunsaturated fatty acids omega-3 and omega-6.

The aim of the study was to assess the efficacy and safety of the use of omega-3/6 on its own and in combination with methylphenidate for the treatment of symptoms of ADHD in a Mexican child population.

The sample had a very similar distribution by sexes and subtypes to that reported in worldwide literature, with a male sex predominance of 2:1 and a higher frequency of the combined subtype in over 50% of the sample. An innovation in this study was the incorporation of the slow, sluggish cognitive tempo subtype into the subtypes to be studied. Although all subtypes responded similarly to the three sets of treatment, a difference was observed in the response of the hyperactivity symptom, which had a greater impact with the use of short-acting methylphenidate, as opposed to the sluggish subtype, which responded better to omega-3/6 being administered. This may be interpreted as a difference between a more right frontal cortical etiology of the hyperactivity and a cerebellar involution of the sluggish subtype. It was also observed that the symptoms of hyperactivity and impulsivity responded better to high doses of short-acting methylphenidate.

One situation to emphasise is that the group combining short-acting methylphenidate with omega-3/6 needed smaller doses of the latter (1.0 mg/kg/day vs. 0.7 mg/kg/day), showing better tolerance to the treatment with fewer reported side-effects. This opens an interesting door onto the initial combination management which may favour not only better tolerance but also better therapy compliance with the combination of drugs.

Additionally, although control and reduction of symptoms is slower in groups B and C, as opposed to group A (short-acting methylphenidate alone), scale scores levelled off towards week 8 of treatment, which suggests long-term stabilisation with combination management. Furthermore, on decreasing the total dosage of methylphenidate, it was observed that fewer effects of anxiety were reported by the patients, including some who had reported signs of anxiety at the start of treatment. After treatment with omega-3/6, these symptoms were reduced. Once patients had achieved improvement (between week 4 and 8 of the treatment), they managed to stay within the treatment, maintaining, in general, good compliance with it. The reasons for this may have multiple explanations, among which is the close monitoring with drug administration support (which, for socio-economic questions in Latin-America, is fundamental), monitoring by a single doctor and drug efficacy. In spite of the fact that in the questionnaire for measuring symptoms of fatty acid deficiency, nobody reported having an increase in these signs, patients reported significant improvement during treatment.

Among the effects on co-morbidities, we can establish that patients in group C displayed a greater impact, particularly in the improvement of the symptoms of specific learning disorders. It is feasible that stabilisation by omega-3/6 of the symptoms of inattention, together with the stimulation of learning paths, are important.

### Conclusions

Further to this study, omega-3 fatty acids were considered to be effective and safe in the treatment of ADHD in paediatric patients and that starting treatment was considered for those patients with ADHD of the inattentive and the slow, sluggish cognitive subtypes, maintaining a dosage of 1.5 g/day and expecting to see favourable responses after 8 weeks of treatment. In combined ADHD and in predominance of hyperactive-impulsive subtypes, especially those with low bodyweight at commencement, the combination of short-acting methylphenidate plus omega-3 fatty acids was considered a good option, managing lower doses of the former and diminishing in this way some of the undesirable effects of the drug.

TABLE 1
Demographic variables means in total and for the respective groups at baseline

| | Total | | Group A | | Group B | | Group C | | p-values |
|---|---|---|---|---|---|---|---|---|---|
| Characteristic | n = 90 | | n = 30 | | n = 30 | | n = 30 | | Between groups |
| Gender, no (%) | | | | | | | | | |
| Male | 61 | (68) | 20 | (66) | 22 | (73) | 19 | (63) | ns |
| Female | 29 | (32) | 10 | (34) | 8 | (27) | 11 | (37) | ns |
| Age, yr | | | | | | | | | |
| mean (SD) | 8.2 | (1.7) | 8 | (1.8) | 8.6 | (1.7) | 8.2 | (1.8) | Ns |
| Weight, kg | | | | | | | | | |
| mean (SD) | 32.7 | (11) | 30 | (8.7) | 36.6 | (12.2) | 32.2 | (11) | p = 0.01 (A vs. B) |
| | | | | | | | | | ns (B vs. C) |
| | | | | | | | | | ns (A vs. C) |
| Height, m | | | | | | | | | |
| mean (SD) | 1.30 | (0.1) | 1.30 | (0.1) | 1.34 | (0.1) | 1.30 | (0.1) | Ns |
| ADHD | | | | | | | | | |
| subtype, no (%) | | | | | | | | | |
| Inattentive | 11 | (12) | 5 | (17%) | 4 | (14%) | 2 | (7%) | ns |
| H-I | 7 | (8) | 2 | (7%) | 3 | (10%) | 2 | (7%) | ns |
| Combined | 51 | (57) | 18 | (59%) | 14 | (46%) | 19 | (63%) | ns |
| sluggish | 21 | (23) | 5 | (17%) | 9 | (30%) | 7 | (23%) | ns |

SD: standard deviation; m: meters

TABLE 2
Side effects in % of the whole population (n = 30) for each group.
Group A: MTP; Group B: Omega 3/6; Group C: Omega3/6 + MTP;

| In % of the subjects | Group A | Group B | Group C |
|---|---|---|---|
| Hiporexia | 53.3 | 6.7 | 26.7 |
| Headache | 23.3 | — | 26.7 |
| Irritability | 16.7 | — | — |
| Tension | 16.7 | — | 6.7 |
| Pallor | 16.7 | — | 16.7 |
| Palpitation | 13.3 | — | 6.7 |
| Sleeplessness | 6.7 | — | — |
| Tics | 6.7 | — | — |
| Tremor | 1.0 | — | — |
| Seasickness | 1.0 | — | — |
| Dyspepsia | — | 30.0 | — |
| Diarrhea | — | 16.7 | — |

TABLE 3
Mean changes in ADHD-RS-SV, Inattentive and Hyperactive/Impulsive subscales at baseline and 12 months for all 3 treatment
groups

| | Group A | | | | | | Group B | | | | | |
|---|---|---|---|---|---|---|---|---|---|---|---|---|
| | | | After 3 | | After 12 | | | | After 3 | | After 12 | |
| | Baseline | | months | | months | | Baseline | | months | | months | |
| | n | Mean | n | Mean | n | Mean | n | Mean | n | Mean | n | Mean |
| ADHD RS, total | 30 | 41.43 ± 4.39 | 24 | 25.29 ± 4.18 | 20 | 23.55 ± 1.72 | 30 | 40.7 ± 4.82 | 29 | 29.07 ± 7.15 | 22 | 23.5 ± 2.84 |
| score | | | | | | | | | | | | |
| Inattentive | 30 | 21.47 ± 3.20 | 24 | 12.04 ± 1.78 | 20 | 11.45 ± 1.32 | 30 | 21.47 ± 3.20 | 29 | 12.96 ± 2.75 | 22 | 10.95 ± 1.00 |
| subscale | | | | | | | | | | | | |
| Hyperactive/ | 30 | 19.93 ± 5.11 | 24 | 13.25 ± 3.05 | 20 | 12.1 ± 1.29 | 30 | 19.37 ± 6.06 | 29 | 16.11 ± 5.41 | 22 | 12.55 ± 2.24 |
| impulsive subscale | | | | | | | | | | | | |

| | Group C | | | | | | | |
|---|---|---|---|---|---|---|---|---|
| | | | After 3 | | After 12 | | ANOVA | |
| | Baseline | | months | | months | | (LOCF) | |
| | n | Mean | n | Mean | n | Mean | F | pvalue |
| ADHD RS, total | 30 | 42.03 ± 3.99 | 29 | 26.96 ± 4.87 | 27 | 22.89 ± 1.76 | 0.715 | 0.493 |
| score | | | | | | | | |
| Inattentive | 30 | 21.57 ± 2.84 | 29 | 12.43 ± 2.04 | 27 | 10.78 ± 0.85 | 2.44 | 0.95 |
| subscale | | | | | | | | |
| Hyperactive/ | 30 | 20.6 ± 5.45 | 29 | 14.46 ± 3.12 | 27 | 12.11 ± 1.42 | 0.502 | 0.607 |
| impulsive subscale | | | | | | | | |

Group A: MTP; Group B: Omega 3/6; Group C: Omega3/6 + MTP

1. Composition for use in treating and/or preventing mental and behavioral disorders and/or improving mental health and cognitive functions in a subject, comprising:
a) Docosahexaenoic acid (DHA) and/or the pharmaceutically acceptable derivatives and/or precursors thereof;
b) Eicosapentaenoic acid (EPA) and/or the pharmaceutically acceptable derivatives and/or precursors thereof;
c) γ-linolenic acid (GLA) and/or the pharmaceutically acceptable derivatives and/or precursors thereof; and
at least a psychostimulant selected among methylphenidate HCl, atomoxetine, amphetamine, gamma-hydroxybutyrate, dextroamphetamine, sibutramine, methylenedioxymethamphetamine, and/or an anxiolytic compound selected among fluoxatine, sertraline, paroxetine, fluoroxamine, citralopram, venlafaxine, bupropion, nefazodone, and mirtazapine.

2. Composition according to claim 1, which further comprises at least one vitamin and/or one or more minerals.

3. Composition of claim 2, wherein the vitamin is selected from the group consisting of vitamin B5, vitamin B6, folic acid, vitamin B12, vitamin E and vitamin D.

4. Composition according to claim 2, wherein the vitamin is vitamin B5.

5. Composition according to claim 2, wherein the mineral is zinc and/or iron.

6. Composition according to any one of the preceding claims, wherein said composition further comprises an antioxidant.

7. Composition according to claim 6, wherein said antioxidant is selected among tocopherols (4 toco), Origanox®, oil extract from oregano (Origanum vulgare), thyme (Thymus vulgaris), rosemary (Rosmarinus officinalis), sage (Salvia officinalis) and clove (Syzygium aromaticum), and/or vitamin C.

8. Composition according to claim 1, wherein said composition is in the form of a formulation suitable for oral administration.

9. Composition according to claim 8, wherein said oral formulation is present in the form of hard or soft capsule, microcapsule, oil, syrup, suspension, solid or semi-solid, tablet, sugar-coated tablets, powder, pellet, film-coated tablet, granule, emulsion, paste, gels, lozenges, gums, or chewable formulation, such as chewing-gum.

10. Composition according to claim 9, wherein said oral formulation is a gastro-resistant formulation or a non gastro-resistant formulation.

11. Composition according to claim 1, wherein EPA, DHA and GLA are present in a ratio of approximately 9:3:1.

12. Composition according to claim 1, wherein the subject is a healthy human subject or a human subject in need of such treatment.

13. Composition according to claim 1, wherein said composition is in the form of a pharmaceutical composition, and comprises a pharmaceutically acceptable vehicle.

14. Composition according to claim 1, wherein said composition is administered as a food supplement.

15. Composition according to claim 1 wherein said composition is administered in a therapeutically effective amount to normal children for use in improving learning abilities and/or cognitive functions.

16. Composition according to claim 1, for use in a method treating and/or preventing behavioral and emotional disorders.

17. Composition of claim 16, wherein said behavioral and emotional disorders are hyperkinetic disorders, conduct disorders, disorders of social functioning, tic disorders, and feeding disorders.

18. Composition of claim 17, wherein said hyperkinetic disorders are disturbance of activity and attention, such as Attention Deficit Hyperactivity Disorder (ADHD) or Attention Deficit Disorder (ADD), hyperactivity, and/or cognitive dysfunction.

19. Composition of claim 16, wherein said behavioral and emotional disorders are disorders of psychological development, such as disorders of speech and language, specific developmental disorders of scholastic skills, specific developmental disorder of motor function, and/or pervasive developmental disorders.

20. Composition of claim 19, wherein said psychological development disorders are autism or Autistic syndrome disorder (ASD), apraxia, dyspraxia, or dyslexia.

21. A method of treating and/or preventing mental and behavioural disorders and/or improving mental health and cognitive functions, comprising administering to a subject in need thereof a composition comprising:
a) eicosapentaenoic acid (EPA) and/or the pharmaceutically acceptable derivatives and/or precursors thereof;
b) docosahexaenoic acid (DHA) and/or the pharmaceutically acceptable derivatives and/or precursors thereo;
c) γ-linolenic acid (GLA) and/or the pharmaceutically acceptable derivatives and/or precursors thereof; and
a psychostimulant compound selected among methylphenidate HCl, atomoxetine, amphetamine, gamma-hydroxybutyrate, dextroamphetamine, sibutramine, methylenedioxymethamphetamine, and/or an anxiolytic compound selected among fluoxatine, sertraline, paroxetine, fluoroxamine, citralopram, venlafaxine, bupropion, nefazodone, and mirtazapine.

22. The method of claim 21, wherein the composition further comprises a vitamin selected from a group comprising vitamin B5, vitamin B6, folic acid, vitamin B12, vitamin E and vitamin D.

23. The method of claim 21, wherein the composition further comprises on or more minerals.

24. The composition of claim 23, wherein the mineral is zinc and/or iron.

25. The method of claim 21, wherein the said composition further comprises an antioxidant.

26. The method according to claim 25, wherein said antioxidant in the composition is selected among tocopherols (4 toco), Origanox, oil extracts from oregano (Origanum vulgare), thyme (Thymus vulgaris), rosemary (Rosmarinus officinalis), sage (Salvia officinalis) and clove (Syzygium aromaticum), and/or vitamin C.

27. The method according to claim 21, wherein said composition is in the form of a formulation suitable for oral administration.

28. The method according to claim 27, wherein said oral formulation is present in the form of hard or soft capsule, microcapsule, oil, syrup, suspension, solid or semi-solid, tablet, sugar-coated tablets, powder, pellet, film-coated tablet, granule, emulsion, paste, gels, lozenges, gums, or chewable formulation, such as chewing-gum.

29. The method according to claim 21, wherein said oral formulation is a gastro-resistant formulation or non gastro-resistant formulation.

30. The method according to claim 21, wherein the subject is a healthy human subject or a human subject in need of such treatment.

31. The method according to claim 21, wherein said composition is administered as a food supplement.

32. The method according to claim 21, wherein said composition is in the form of a pharmaceutical composition, and comprises a pharmaceutically acceptable vehicle.

33. The method according to claim 21, wherein said adult in need of such treatment are subject to mental and behavioral disorders.

34. The method according to claim 21, wherein EPA, DHA and GLA in the said composition are present in a ratio of approximately 9:3:1.

35. The method according to claim 21, wherein said composition is administered in a therapeutically effective amount to normal children for use in improving learning abilities and/or cognitive functions.

36. The method according to claim 21, wherein said composition is administered in a therapeutically effective amount to children for treating and/or preventing behavioral and emotional disorders.

37. The method according to claim 36, wherein said behavioral and emotional disorders are hyperkinetic disorders, conduct disorders, disorders of social functioning, tic disorders, and feeding disorders.

38. The method according to claim 37, wherein said hyperkinetic disorders are disturbance of activity and attention, such as Attention Deficit Hyperactivity Disorder (ADHD) or Attention Deficit Disorder (ADD), hyperactivity, or cognitive dysfunction.

39. The method according to claim 37, wherein said behavioral and emotional disorders are disorders of psychological development, such as disorders of speech and language, specific developmental disorders of scholastic skills, specific developmental disorder of motor function, pervasive developmental disorders

40. The method according to claim 39, wherein said psychological development disorders are autism or Autistic syndrome disorder (ASD), apraxia, dyspraxia, or dyslexia.
